(19) United States
(12) Patent Application Publication
(10) Pub. No.: US 2014/0194515 A1
(43) Pub. Date: Jul. 10, 2014

(54) Use of a Composition Comprising an Antacid, a Mucolytic and a Gelling Agent in the Treatment of Cold

(71) Applicant: Barbara Nelson, Karlskoga (SE)

(72) Inventor: Barbara Nelson, Karlskoga (SE)

(21) Appl. No.: 14/153,701

(22) Filed: Jan. 13, 2014

Related U.S. Application Data
(63) Continuation of application No. 11/992,762, filed on Mar. 28, 2008, now abandoned.

(30) Foreign Application Priority Data
Sep. 29, 2005 (SE) 0502133.2

(51) Int. Cl. A61K 31/198; A61K 47/36; A61K 45/06

(52) U.S. Cl. CPC A61K 31/198; A61K 45/06; A61K 47/36
USPC 514/562

(57) ABSTRACT

The present invention is directed to a medicine to alleviate and cure colds by reducing acid content and stimulation of increased mucus production due to the fact that active components of the substance consist of acid stanching components like proton pumps stanches, histamine-2-receptor-antagonists, mucus stimulating components as acetylcysteine and alginic acid. This together creates an optimal healing environment in the respirator passages.

The cause for colds and other respiratory diseases may be the consequence 008 of a long cold period.

Colds are infections causing viruses. There are about 200 cold viruses constantly around us, the most usual are rhinoviruses. To enable the cold virus to enter the mucous membranes of the respiratory passages the mucous membrane must be injured, have a micro sore, through which the virus can enter into the cell and propagate itself. The patient gets a cold. Like nasal catarrh, a sore throat, cough, headache and a slight fever. Why does one get a cold? Not everybody gets a cold. Why? Well, a complete undamaged mucous membrane is not liable to virus and bacteria. What makes the mucous membrane liable? The mucous membrane gets micro sores. Micro sores are caused by aerosol of the hydrochloric acid from the stomach which is pressed upwards due to increased abdominal pressure, overweight, heavy work, hereditary, insufficient upper orifice of the stomach, latter can depend on mineral shortage, such as calcium and magnesium. Micro sores cause inflammation of the mucous membrane, i.e. slightly redness and swelling which means an inflammation. When virus and/or bacteria enter the mucous membrane it will be infected, leading to increased redness, swelling and moistening (a cold). Once the mucous membrane is inflamed there is a big risk for infection. The inflammation depends on a weakness of the concordance between the different functions of the body. The amount hydrochloric acid, and in combination with insufficient upper orifice of the stomach, is of importance. The quantity in the stomach is, however, vital for the digestion. A good nutrition absorption results in good health and resisting power but for certain persons this causes problems. An adult produces in average 2 to 3 litres of strong acid gastric juice each day. Undiluted gastric juice has a pH-value of 1.8. The hydrochloric acid becomes aerosol due to quantity and abdominal pressure. Each time in connection with physical strain in combination with insufficient upper orifice of the stomach, for example, each time one sits down and gets up again or lies down, the hydrochloric acid-aerosol is pressed up the esophagus. This irritates the pharynx and the mucous membrane of the nose and during inhalation it returns down the trachea and bronchial tubes and irritates the mucous membrane which then produces more mucus as usual in order to protect the mucous membranes from the caustic effect caused by the hydrochloric acid-aerosol. We have now a viral infection which often comes first. If the immune defence of the body is unable to heal a cold, and may take two (2) weeks or more. The bacteria can easily take over if not the hydrochloric acid-aerosol problem of the patient is treated with inhibiting medicaments (see patent claim). The hydrochloric acid-aerosol causes dehydration of the mucous membrane in the respiratory passages. At first they get micro sores. The mucous membrane defends itself with white blood cells and increased production of secret which will be increasingly discoulored as the infection is getting worse. From clear to white, to green and at last yellow. Sometimes it is mingled with blood. (sinuit). Description of symptoms of a beginning cold. At stadium 1 irritation of the pharynx, a sore throat, a running nose, possible sneezing, increased mucus production. The patient often clears its throat, has a slightly irritating cough, intermittent ear, sometimes impaired hearing, intermittent hoarseness, “a frog in his throat”.

### Further Development of the Cold.

Stadium II. After some week, depending on the quantity hydrochloric acid and how insufficient the orifice of the stomach is, further depending on the carriage of the body, working posture, physical activity and may be overweight. Sore throat, dry throat (risk for tonsillitis), nasal catarrh, nasal congestion, headache (risk for sinusitis, intermittent ear and impaired hearing due to suppression in the middle ear (the muzzle of the auditory tube in the upper pharynx is swelling and no air is coming up in the middle ear when swallowing and the tympanic membrane/-curves in due to vacuum. Liquid is formed inside the tympanic membrane (/-es) and get more or less immobile. Pain follows and a risk for impaired hearing and otitis, hoarseness (risk for laryngitis), irritating cough, which increases during night, disturbed sleep, tiredness (risk for pneumonia), increased coughing, coughing attacks, feeling of vomiting. These patients get often up at night, eat and drink as it relieves the pain. The patient increases in weight (which increases insufficiency in the upper orifice of the stomach and thus increased snoring problem and more and more tired and higher sensitivity for infections. Risk for high blood pressure and the patient will be dangerous to traffic. The mucous membrane is now hypersensitive to hydrochloric acid-aerosol (also to strong scent, for example, perfume, tobacco-smoke, car exhaust fumes etc.). Each time a patient is coughing, his abdominal pressure will increase and therefore the risk for hydrochloric acid-aerosol in the respiratory passages increases. The patient has entered a bad circle. When the patient is in the second stadium, the mucous membrane cannot produce enough protecting mucus. At first increases the bleeding in the mucous membrane which gets red and swollen. If the hydrochloric acid-aerosol irritation continues for a longer period, there will be a vasodilator in the back part of the pharynx and at the same time the mucous membrane gets bleak and thick. Both stadium 1 and 2 cause hydrochloric acid-aerosol micro sores in the mucous membrane which create a gateway for, primarily, virus and also for bacteria and there will be an infection of the respiratory passages. The hydrochloric acid-aerosol does not only destroy the mucous membranes but also the cilia. These facts contribute that the allergens can enter into the blood and create allergy to the patients who have the genes for it. The mucous membranes are swelling and make the lumen of the respiratory passages minor and furthermore, there will be micro sores in the mucous membrane where the virus and bacteria easily can attack. This will cause the following.

### Asthma

Why do certain athletics get asthma? Some sporting youths which are jogging, skiing, making sit-ups etc., get irritating cough-asthmatic problems and get very tired with the least effort. Their physical effort increases the abdominal pressure. If they have the upper orifice of the stomach-insufficiency, the hydrochloric acid-aerosol goes up and down the throat and will be entering the lungs after inhaling—the mucous membrane in the trachea and bronchi swell and contract, there will be cough and asthma. These patients are treated with bronchial dilatation which has a good effect. Adrenergic: Beta-stimulators widen the bronchi, also increase the muscle tonus in the upper orifice of the stomach and lower the hydrochloric production too. As inflammation stanch the patients get cortisone as inhalation medicine sometimes water solving tablets and sometimes, but rather seldom, injections. With troublesome mucus production the patient gets anti-chronic medicine which reduces the mucus production and also the hydrochloric acid production. Thanks to the development of medicine against asthma the visitors to our care centres have been reduced during the last ten years. I can also notice that the visits coincide with the visit periods of some of our stomach catarrh patients. I mean, that there are these 6 to 8 periods a year when the stomach, out of some reason, produces more hydrochloric acid as usual and causes increased problems for patients with asthma and chronicle bronchitis. It would help these patients a lot if they could be treated with hydrochloric acid-aerosol stanching medicine.

### Chronicle Bronchitis

Why do certain persons get chronicle bronchitis? Chronicle bronchitis is not only seen with smokers but also with middle aged men and women with overweight. Overweight results in increased abdominal pressure, especially when they are sitting (often sank down). They feel well when they eat and drink and thus they get even more overweight. When they sleep the upper orifice of the stomach relaxes, ventral fatness creates increased abdominal pressure, hydrochloric acid-aerosol goes up and irritates the respiratory passages, irritates to cough, some get coughing attacks, which actually is a defence against the hydrochloric acid-aerosol and increased mucus production. Longer periods of hydrochloric acid-aerosol exposition create micro sores in the mucous membrane and these people do often get respiratory infections (among others of sinuites, broncho pneumonias). Besides the fact that chronicle bronchitis causes overweight, it gets narrow in the throat and the patient becomes snoring problems. The hydrochloric acid-aerosol increases the snoring problems essentially and they will get dyspnea and be extremely tired. Furthermore they will be a danger to the traffic. They quite often suffer from high blood pressure.

### Chronicle Obstructive Pneumonia.

The hydrochloric acid-aerosol according to the above mentioned, plus smoking and/or grinding dust, stone dust, micro fibre from mineral wool damages the mucous membrane in the respiratory passages. The cilia stop gradually to work. In the beginning increased mucus production but ones the mucous membrane is damaged the viscosity of the mucous is changed and the mucous membranes cannot protect themselves from harmful micro agents in the inhaled air, which else is transported up together with the mucus with the aid of the cilia to the throat to be swallowed. If unbalance arises in the, for our lungs vital continuous humectant mucus production, the cilia will not function either and the transport of foreign agents to the throat will fail. A chronicle inflammation will cause destruction of the elastic components in the pulmonary tissue, the respiratory passages will easier collapse. These patients get easily dyspnea at minor effort and are very susceptible to infections. The function of the lung gets gradually worse. By reducing the hydrochloric acid-aerosol and thus the inflammation, it is possible to reduce the sensitiveness to infections and hopefully the negative progress. It should be added that the respiration for these patients demand such a lot of energy that they lose weight and really get thin.

### Children With Constantly Returning Respiratory Infections

Certain babies are constantly having a cold or often returning colds with healthier periods of 3 weeks. Reasons: Underdeveloped upper orifice of the stomach, degree of maturity, the smooth upper muscles of the orifice of the stomach does not work properly due to shortage of minerals. Calcium and magnesium (valid for adults too). Children who are at the breast do have a much lower frequency of respiratory infection, mostly because they get the quantity of calcium and magnesium from their mother. Especially the calcium need is big for the growing child's skeleton. Naturally breastfeeding gives many antibodies, also as protection against infections. The flat position in bed increases the hydrostatic pressure and increases in this way the mucous membrane swells and with simultaneous insufficiency of the upper orifice of the stomach the hydrochloric acid-aerosol turns up the upper respiratory passages in order to return down the bronchial tubes when inhaling. The mucous membranes in the bronchial tubes react by inflammation symptoms: Increased mucus production and coughing attacks (which is a defence against the hydrochloric acid-aerosol). There is a risk that the hydrochloric acid-aerosol cause micro sores in the mucus membranes and by that a risk for infection, for example, pneumonia. This circumstance causes also a swollen mucous membrane in the osties of the auditory tubes (exits) in the pharynx (upper throat), and thus the respiratory passage to the inner ear does not work satisfactory during the swallowing procedure. Then the air in the inner ear is absorbed, there will be a vacuum and gradually there will be formed a liquid behind the eardrum (or drums) which is a breeding ground for growth of bacteria and infections. Otitis If high bedding does not help, lift up the bed on the head end (the hammock of the “primitive people” is a good idea) it would be suitable with a hydrochloric acid-aerosol stanching medicine. Those children with constant running noses and cough come into a bad circle. The have a poor sleep, are exhausted, do not want to eat, do not grow, are sensitive to infections, stressed and whimpering. They get increased hydrochloric acid product or have too much hydrochloric acid in comparison with meals. The visceral content of their stomach gets extra sour and the intestine works faster to get rid of the sour intestine irritating content—the get loose sour evacuation of the bowels—bad nutritional absorption and risk for dehydration. Those children do easily catch asthma, especially if they have the genes for it. They would feel much better if they would get hydrochloric acid-aerosol stanch medicine and probably totally recover.

According to above written study, hydrochloric acid-aerosol stanch medicine, according to patent demands, has very good effect on treatments of upper infection of the respiratory passage (cold). The treatment must start early when symptoms occur like tickling, throat itching, nose irritation, it hurts when swallowing and thus reduce the risk for the following illness to develop into complication as cold, asthma chronicle bronchitis and chronic obstructive pneumonia.

Hydrochloric acid-aerosol stanch medicine has also good effect on constantly returning respiratory passage infections and infections on children. With constantly returning laryngitis, throat infections and broncho pneumonia early treatment of colds is vital. It has also good effect on stressful throats. Intermittent hoarseness, rasping throat, hawkings. Snoring troubles which do not respond to high bedding or weight reduction.

Cold periods, 6-8 times/year, affect most of us. Coriza virus exist all year-round and constantly in our environment but something it is affecting our body to produce more hydrochloric acid in intervals of 6-8 times/year and we are getting a cold. After studying the patients and their case books I have noticed, that gastric catarrh and colds coincide from year to year during the same time and interval, as well as increasing trouble and infections of asthmatic patients and patients with chronicle bronchitis and chronic obstructive pneumonia. According to my study I claim that hydrochloric acid-aerosol stanching medicine has immediate protecting effect on the mucous membranes of the respiratory passages. Thus they are not easily liable to bacterial and non-bacterial inflammation. Out of this reason it is possible to reduce the use of antibiotics considering the increased development of the resistance and thus reduce the use of cortisone for non-bacterial inflammation as asthma, chronicle bronchitis and chronic obstructive pneumonia. These patients with asthma, chronicle bronchitis and chronic obstructive pneumonia get often long-term treatment of medicine with cortisone content, which is harmful but, of course, it must be well-balanced in each case. Long-term treatment with cortisone is harmful, creates adrenal cortex insufficiency of the glands, high blood pressure, decalcification of the skeleton, diabetes, skin fragility, bad healing, gastric ulcer, risk for intestinal hemorrhage etc. and should not be prescribed for children due to inhibition of growth, not to pregnant women either. With hydrochloric acid-aerosol stanching medicine conditions are created for fast healing of the mucous membranes, as the harmful hydrochloric acid-aerosol no longer can affect the mucous membranes. The present treatment of infection of the respiratory passage: Pain-killing, fever lowering, nose drops, expectorant cough syrup, medicine that relieves cough and pain-relieving throat pastilles. For patients with high fever which seem to have a bacterial infection, antibiotics will be subscribed. The latter we wish to avoid thinking of the increased antibiotic-resistance.

In view of my clarification I claim that a cold should be treated with acid stanching, algine acid, acetylsystein and minerals. A cold is cured by creating an optimal healing environment and preventing development of bacterial infections (reduction of the use of antibiotics and less antibiotic resistant pathogenic bacteria) and chronicle diseases in respiratory passages. According to patent demands acid stanching reduces the quantity hydrochloric acid in the stomach, as proton pump stanching, histamine 2-receptor antagonists. They reduce the production of hydrochloric acid and to a certain degree the quantity fluid in the stomach, however, not enough to prevent the hydrochloric acid-aerosol to pass the insufficient orifice of the stomach (see above), but it has to be supplemented with algine acid which adheres like a jelly in the upper part of the stomach and prevents the hydrochloric acid-aerosol to reach the respiratory passages.

(Proton pump stanching and histamin-2 antagonists are examples of acid stanching means which reduce the production of hydrochloric acid in the stomach. This is what I mainly wish to attain.

### Patent Demand for Other Medical Indications:

The use of a composition for preparation of a medical product for treatment and healing of cold, see below. An acid stanch. to reduce the quantity hydrochloric acid in the stomach as proton pump stanch, histamin-2-receptor antagonists. They reduce production of hydrochloric acid and to a certain degree quantity fluid in the stomach, however, not enough to prevent the hydrochloric acid-aerosol to pass the insufficient orifice of the stomach but it has to be supplemented with algine acid. Algine acid adheres like a jelly in the upper part of the stomach and prevents the hydrochloric acid-aerosol to reach the respiratory passages. Acethylsystein: Mucolythicum, mucus adjusting. Expextorant, changes the viscosity of the mucus by splitting proteins, increases the mucus quantity by diluting (watery). The mucus gets diluted and is thus able to rinse out the mucus membranes. For that reason the expression mucus stimulating. Calcium and magnesium are minerals which are integrated in the task of the muscle cells and influence the muscle tonus. In this connection it is of importance for the upper orifice of the smooth muscles of the stomach to make it close better. See part “Children with constantly returning infection of the respiratory passages”. It is also a good stabilizer.

### Treatment Dose:

The dose is adapted to age, weight and physiological status. The dosage is adapted to choice of acid stanch if one chooses a proton pump stanch or histamin-2-antagonist or any other acid stanch. (It depends on the pharmaceutical manufacturer that will take care of it. Preferably a company that manufactures acid stanches. The pharmaceutical manufacturer will probably make a double-blindfold test. After that the medicine must be approved by the Medical Products Agency.

### EXAMPLE

Adult. Time of treatment: 3-4 days. Proton pump stanch: Acid resistant granules solved in the intestine 40 mg/twenty four hours, divided on 2 times//twenty four hours. Mucolythicum, mucus adjusting. Acetylsystein 150 mg 4 times//twenty four hours. Algine acid: 1 g 4 times//twenty four hours. Minerals: Calcium 250 mg 4 times//twenty four hours. Magnesium 60 mg.

4 times//twenty four hours/twenty four hours.

It is known that inflammation of the respiratory passages in the mucous membranes is treated with medicine, especially intended for respiratory organs. There has also been taken into consideration in what way there may be a connection between specific states of ill-health in the respiratory passages and the returning flow of the gastric juice from the stomach to the oesophagus and therefore used the well-known and frequently used acid depressing medicine against gastro reflux (GERD).

A fact, which has not been known earlier, is that the acidity of the stomach turns to aerosol which arises from the increased abdominal pressure in combination with bad muscle tonus in the smooth muscles in the upper orifice of the stomach which, among others, depends on low calcium and magnesium in combination with physical strain, overweight, flat position, stress, inheritance, and not to forget the time cycles (6-8 times/year).

The hydrochloric acid-aerosol damages, in the first place, the mucous membranes in the upper respiratory passages (see description). The mucous membranes get micro sores which are the gateway for mainly virus and the consequence will be that the patients will suffer from colds. If the cold is lengthy, it often causes a bacterial infection such as sinusitis, otitis, tonsillitis, laryngitis and pneumonia. Some patients, especially those who are predisposed due to hereditariness, are easily affected by chronic states of illness in the respiratory passages due to frequently returning cold. (See description).

The aim of the present invention is to reduce poor health and at the same time reduce the use of antibiotics which in its turn leads to smaller amounts of antibiotic strain of bacteria. In other words, inventions aid public health in the world.

1-4. (canceled)

5. A composition comprising an antacid, a mucolytic and a gelling agent.

6. The composition of claim 5, wherein the antacid is selected from the group consisting of a proton pump inhibitor, a histamine-2-receptor antagonist, and a sucralate.

7. The composition of claim 5, wherein the mucolytic is acetylcysteine.

8. The composition of claim 5, wherein the gelling agent is alginic acid.

9. The composition of claim 8, wherein the alginic acid is derived from algae.

10. The composition of claim 5, further comprising calcium and magnesium salts, and stabilizing agents.

11. The composition of claim 5, further comprising a flavoring.

12. A method of treating and/or preventing and/or curing a cold, said method comprising administering to a patient in need thereof an effective amount of the composition of claim 5.
